(19) United States
(12) Patent Application Publication
(10) Pub. No.: US 2010/0168030 A1
(43) Pub. Date: Jul. 1, 2010

(54) Methods and compositions for treatment of organophosphate-caused pathology

(76) Inventors: Csaba K. Zoltani, Lutherville, MD (US); Gennady E. Platoff, Millersville, MD (US); Steven I. Baskin, Bel Air, MD (US)

Correspondence Address: U S ARMY RESEARCH LABORATORY;ATTN: RDRL-LOC-I, 2800 POWDER MILL RD, ADELPHI, MD, 20783-1197, US

(21) Appl. No.: 11/288,269

(22) Filed: Nov. 29, 2005

(51) Int. Cl. A61K 38/16; A61P 9/06

(52) U.S. Cl. 514/13

(57) ABSTRACT

A method of treating an organophosphate toxin-caused cardiac abnormality is described which includes administering a pharmaceutical composition including a therapeutically effective amount of a chloride current modulator to an individual subject having a cardiac abnormality caused by intoxication with an organophosphate toxin. The chloride current modulator is effective to modulate a chloride conductance and thereby reduce a symptom or sign of an organophosphate toxin-caused cardiac abnormality, thus treating the toxin-caused cardiac abnormality. Optionally, included is administering a therapeutic agent to inhibit an organophosphate toxin-caused distortion of the action potential of myocytes, support restoration of usual intracellular ionic concentrations, and support an increase in cardiac contractility. A composition according to the invention is described which includes a chloride current modulator; and a therapeutic agent to inhibit an organophosphate toxin-induced distortion of the action potential of myocytes, support restoration of usual intracellular ionic concentrations, and support an increase in cardiac contractility.

### GOVERNMENT INTEREST

The invention described herein may be manufactured, used, and licensed by or for the United States Government.

### FIELD OF THE INVENTION

This invention relates to compositions and methods for treatment of organophosphate poisoning. In particular, the invention relates to compositions and methods for treatment of cardiac arrhythmias and/or repolarization abnormalities resulting from organophosphate poisoning.

### BACKGROUND OF THE INVENTION

Organophosphates are widely used as insecticides and as cholinergic threat agents. Accidents in their use are common in agriculture and treatment is still rudimentary. Organophosphate-caused ventricular fibrillation results in heart failure and is recognized as the most common cause of mortality in cases of organophosphate intoxication.

The first organophosphate toxins were developed early in the 20^{th} century, primarily as pesticides. In the course of research on their pesticidal properties, the potential of these agents for use in chemical warfare and/or as threat agents was discovered.

Organophosphate toxins bind to acetylcholinesterase, preventing the hydrolysis of acetylcholine and leading to its accumulation and consequent prolongation of its effects at synapses. Symptoms of organophosphate toxin intoxication include rhinorrhea, dyspnea, sweating, miosis, bradycardia, tachycardia, loss of consciousness, convulsions, flaccid paralysis, and apnea. Histological examination of organophosphate-affected heart tissue shows cell swelling, hemorrhaging and necrosis.

One of the most serious consequences of organophosphate intoxication is due to effects on the cardiovascular system. Organophosphate-caused heart failure is a syndrome characterized primarily by left ventricular dysfunction, reduced exercise tolerance, impaired quality of life and dramatically shortened life expectancy. Decreased contractility of the left ventricle leads to reduced cardiac output with the consequent systemic arterial and venous constriction.

Cardiovascular effects of organophosphate intoxication are apparent when analyzed by electrocardiogram. The electrocardiogram of an affected individual shows changes in the P-wave, the depolarization of the atria, and also prominent modulation of the T-wave, the repolarization of the ventricles. After an initial bradycardia, Torsade de Pointes and tachycardia develop and the electrophysiology of the heart loses its predictability. In addition to these changes, the fast outward rectifying potassium current is blocked. The ensuing over-stimulation results in ion concentration imbalance and membrane current derangement. On the electrocardiogram, this is expressed by QT prolongation, ST and T wave abnormalities. Organophosphate-caused ventricular fibrillation culminates in heart failure and is recognized as the most common cause of mortality in cases of organophosphate intoxication.

An example of effects of organophosphate compounds on cardiac tissue is shown in FIG. 1 which illustrates a simulated electrocardiogram (ECG or EKG) illustrating a control trace, A, representative of normal cardiac activity, and a trace B illustrating differences due to organophosphate. A normal T-wave is shown in the simulated control trace, A, at (a). Notable changes include a decrease in amplitude of the QRS and the modulation of the T-wave, the repolarization of the ventricle. In particular, a T-wave as a separate entity is no longer in evidence, the shape of the QRS portion has been altered and the peak has been shortened.

Treatment of organophosphate intoxication has focused predominantly on treatment of symptoms and clearance of organophosphate compounds from the body. Particular treatments include administration of atropine and anisodamine for treatment of symptoms such as gastrointestinal and/or respiratory distress. Side effects may include arrhythmia at higher doses. Further, particular oximes including obidoxime, pralidoxime and asoxime may be used in organophosphate-intoxicated patients to help restore acetylcholinesterase activity.

Thus, there is a continuing need for new treatments for organophosphate poisoning and for treating conditions relating to organophosphate exposure. In particular, treatments addressing cardiac sequelae of organophosphate exposure and other types of toxin intoxication are especially sought after.

### SUMMARY OF THE INVENTION

A method of treating an organophosphate toxin-induced cardiac abnormality in an individual subject is described which includes administering a pharmaceutical composition including a therapeutically effective amount of a chloride current modulator to an individual subject having a cardiac abnormality caused by intoxication with an organophosphate toxin. The chloride current modulator is effective to modulate a chloride conductance and thereby reduce a symptom or sign of an organophosphate toxin-induced cardiac abnormality, thus treating the toxin-induced cardiac abnormality.

In a preferred option, the chloride current modulator is a modulator of I_{Cl, swell}. Modulators of chloride currents include: a disulfonic stilbene; an arylaminobenzoate; a fenamate; an anthracene carboxylate; an indanylalkanoic acid; a clofibric acid; a clofibric acid derivative; a sulfonylurea; a calixarene; suramin; and tamoxifen. Further preferred are modulators of I_{Cl, swell} such as 4,4′-diisothiocyanostilbene-2,2′-disulfonic acid (DIDS); 4,4′-dinitrostilbene-2,2′-disulfonic acid (DNDS); 4-acetamindo-4′-isothiocyanostilbene-2,2′-disulfonic acid (SITS); tamoxifen; 5-nitro-2-(3-phenylpropylamino)benzoic acid (NPPB); niflumic acid (NFA); flufenamic acid; anthracene-9-carboxylate (9AC); diphenylaminecarboxylate (DPC); 2-(p-chlorophenoxy)propionic acid (CPP); and indanyloxyacetic acid (IAA-94). Mixtures of these modulators may also be administered.

Optionally, an inventive method further includes administering a therapeutic agent to inhibit organophosphate-induced distortion of the action potential of myocytes, support restoration of usual intracellular ionic concentrations, and support an increase in cardiac contractility. Such therapeutic agents include a second current modulator; an antiarrhythmic drug; a modulator of a mitochondrial membrane ion channel, ion pump and/or ion exchanger; inhibitor of protein kinase C; an anticonvulsant; an organophosphate clearing agent; an inhibitor of a muscarinic potassium channel; and an acetylcholine receptor antagonist; and combinations thereof.

A composition according to the invention is described which includes a chloride current modulator; and a therapeutic agent to inhibit organophosphate-induced distortion of the action potential of myocytes, support restoration of usual intracellular ionic concentrations, and support an increase in cardiac contractility. In a preferred option, the chloride current modulator is a modulator of I_{Cl, swell}. Further optionally, the therapeutic agent included in an inventive composition is selected from the group consisting of: a second current modulator, an antiarrhythmic drug, an acetylcholine receptor antagonist, an anticonvulsant, an organophosphate clearing agent, an inhibitor of protein kinase C, a modulator of a mitochondrial membrane moiety, and a combination thereof.

### BRIEF DESCRIPTION OF THE DRAWINGS

FIG. 1 shows a simulated electrocardiogram (ECG or EKG) illustrating a control trace, A, representative of normal cardiac activity, and a trace B illustrating differences due to organophosphate; and

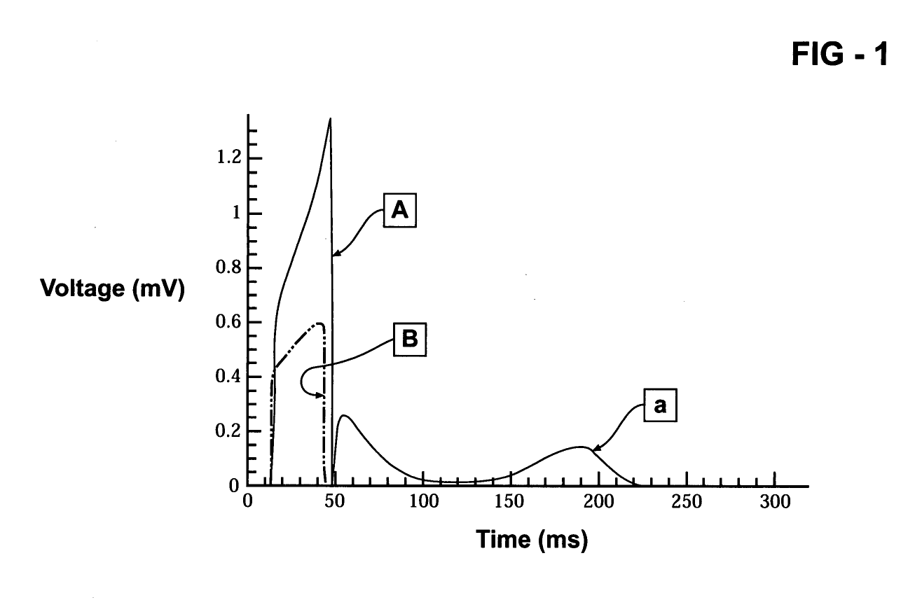

FIG. 2 is a graph showing simulated action potentials and changes thereof in various states relating to organophosphate intoxication.

### DETAILED DESCRIPTION OF THE PREFERRED EMBODIMENTS

A method of treating a toxin-induced cardiac abnormality in an individual subject is provided which includes administering a pharmaceutical composition containing a therapeutically effective amount of an ion channel modulator to an individual subject having a cardiac abnormality caused by intoxication with a toxin. The ion channel modulator is effective to modulate an ion channel conductance, thereby reducing a symptom or sign of a toxin-induced cardiac abnormality and treating the toxin-induced cardiac abnormality.

In a particular embodiment, an inventive method is a method of treating an organophosphate toxin induced cardiac abnormality. An organophosphate toxin is an organophosphate compound which is a pesticide and/or chemical warfare agent having acetylcholinesterase inhibitor activity. Acetylcholinesterase inhibitor activity may be assayed by any of various methods known in the art. For example, an Ellman assay may be used to detect acetylcholinesterase inhibitor activity of an organophosphate pesticide or nerve agent toxin. Briefly described such an assay involves addition of human acetylcholinesterase in an appropriate buffer, such as 100 mM sodium phosphate buffer, pH 7.4, to an organophosphate pesticide or nerve agent toxin of interest. A substrate for acetylcholinesterase such as acetylthiocholine is added to the mixture along with Ellman's reagent 5′5-dithio-bis-(2-nitrobenzoate). After a brief incubation, optical density is measured at 415 nm where optical density is inversely proportional to the inhibiting activity of the organophosphate pesticide or nerve agent toxin (Ellman, G. L. et al., Biochemical Pharmacology, 7: 88-95, 1961 and Wilson, B. W. et al., International Journal of Toxicology, 21: 385-388, 2002).

Many organophosphate toxins are known and are typically esters, amides, or thiol derivatives of phosphoric, phosphonic, phosphorothioic, or phosphonothioic acids.

Organophosphate toxins which are pesticides illustratively include bromfenvinfos (2-bromo-1-(2,4-dichlorophenyl)ethenyl diethyl phosphate), chlorfenvinphos (2-chloro-1-(2,4-dichlorophenyl)ethenyl diethyl phosphate), crotoxyphos (1-phenylethyl3-(dimethoxyphosphinoyloxy)isocrotonate), dichlorvos(2,2-dichlorovinyl dimethyl phosphate), dicrotophos ((E)-2-dimethylcarbamoyl-1-methylvinyl dimethyl phosphate or 3-dimethoxyphosphinoyloxy-N,N-dimethylisocrotonamide), dimethylvinphos ((Z)-2-chloro-1-(2,4-dichlorophenyl)vinyl dimethyl phosphate), fospirate (dimethyl 3,5,6-trichloro-2-pyridyl phosphate), heptenophos(7-chlorobicyclo[3.2.0]hepta-2,6-dien-6-yl dimethyl phosphate), methocrotophos ((E)-2-(N-methoxy-N-methylcarbamoyl)-1-methylvinyl dimethyl phosphate) or (3-dimethoxyphosphinoyloxy-N-methoxy-N-methylisocrotonamide), mevinphos ((EZ)-2-methoxycarbonyl-1-methylvinyl dimethyl phosphate), monocrotophos (dimethyl (E)-1-methyl-2-(methylcarbamoyl)vinyl phosphate), naftalofos (diethyl naphthalimido-oxyphosphonate), phosphamidon ((EZ)-2-chloro-2-diethylcarbamoyl-1-methylvinyl dimethyl phosphate), propaphos (4-(methylthio)phenyl dipropyl phosphate), TEPP (tetraethyl pyrophosphate), and tetrachlorvinphos ((Z)-2-chloro-1-(2,4,5-trichlorophenyl)vinyl dimethyl phosphate); organothiophosphate pesticides include: dioxabenzofos ((RS)-2-methoxy-4H-1,3,2λ5-benzodioxaphosphinine 2-sulfide), fosmethilan (S—[N-(2-chlorophenyl)butyramidomethyl]O,O-dimethyl phosphorodithioate), and phenthoate (S-α-ethoxycarbonylbenzyl O,O-dimethyl phosphorodithioate); aliphatic organothiophosphate pesticides such as: acethion (S-(ethoxycarbonylmethyl) O,O-diethyl phosphorodithioate), amiton (S-2-diethylaminoethyl O,O-diethyl phosphorothioate), cadusafos (S,S-di-sec-butyl O-ethyl phosphorodithioate), chlorethoxyfos (O,O-diethyl(RS)—O-(1,2,2,2-tetrachloroethyl)phosphorothioate), chlormephos (S-chloromethyl O,O-diethyl phosphorodithioate), demephion (reaction mixture of O,O-dimethyl O-2-methylthioethyl phosphorothioate and O,O-dimethyl S-2-methylthioethyl phosphorothioate), demeton (reaction mixture of O,O-diethyl O-2-ethylthioethyl phosphorothioate and O,O-diethyl S-2-ethylthioethyl phosphorothioate), disulfoton (O,O-diethyl S-2-ethyl thioethyl phosphorodithioate), ethion (O,O,O′,O′-tetraethyl S,S′-methylene bis(phosphorodithioate)), ethoprophos (O-ethyl S,S-dipropyl phosphorodithioate), IPSP (S-ethylsulfinylmethyl O,O-di-isopropyl phosphorodithioate), isothioate (S-2-isopropylthioethyl O,O-dimethyl phosphorodithioate), malathion (diethyl (dimethoxythiophosphorylthio)succinate), methacrifos (methyl (E)-3-(dimethoxyphosphinothioyloxy)-2-methylacrylate), oxydemeton (methyl (S-2-ethylsulfinylethyl O,O-dimethyl phosphorothioate), oxydeprofos ((RS)-S-2-ethylsulfinyl-1-methylethyl O,O-dimethyl phosphorothioate), oxydisulfoton (O,O-diethyl S-2-ethylsulfinylethyl phosphorodithioate), phorate (O,O-diethyl S-ethylthiomethyl phosphorodithioate), sulfotep (O,O,O′,O′-tetraethyl dithiopyrophosphate), terbufos (S-tert-butylthiomethyl O,O-diethyl phosphorodithioate), and thiometon, (S-2-ethylthioethyl O,O-dimethyl phosphorodithioate); aliphatic amide organothiophosphate pesticides including: amidithion (S-2-methoxyethylcarbamoylmethyl O,O-dimethyl phosphorodithioate), cyanthoate (S—[N-(1-cyano-1-methylethyl)carbamoylmethyl]O,O-diethyl phosphorothioate), dimethoate (O,O-dimethyl S-methylcarbamoylmethyl phosphorodithioate), ethoate-methyl (S-ethylcarbamoylmethyl O,O-dimethyl phosphorodithioate), formothion (S-formyl(methyl)carbamoylmethyl]O,O-dimethyl phosphorodithioate), mecarbam (S-(N-ethoxycarbonyl-N-methylcarbamoylmethyl) O,O-diethyl phosphorodithioate), omethoate (O,O-dimethyl S-methylcarbamoylmethyl phosphorothioate), prothoate, O,O-diethyl S-isopropylcarbamoylmethyl phosphorodithioate), sophamide, S-methoxymethylcarbamoylmethyl O,O-dimethyl phosphorodithioate), and vamidothion (O,O-dimethyl S—(RS)-2-(1-methylcarbamoylethylthio)ethyl phosphorothioate); oxime organothiophosphate pesticides illustratively include: chlorphoxim (2-(2-chlorophenyl)-2-(diethoxyphosphinothioyloxyimino)acetonitrile, phoxim (O,O-diethyl α-cyanobenzylideneamino-oxyphosphonothioate), and phoxim-methyl (O,O-dimethyl α-cyanobenzylideneamino-oxyphosphonothioate); heterocyclic organothiophosphate pesticides such as: azamethiphos (S-6-chloro-2,3-dihydro-2-oxo-1,3-oxazolo[4,5-b]pyridin-3-ylmethyl O,O-dimethyl phosphorothioate), coumaphos (O-3-chloro-4-methyl-2-oxo-2H-chromen-7-yl O,O-diethyl phosphorothioate), coumithoate (O,O-diethyl O-(7,8,9,10-tetrahydro-6-oxo-6H-benzo[c]chromen-3-yl)phosphorothioate), dioxathion (S,S′-(1,4-dioxane-2,3-diyl) O,O,O′,O′-tetraethyl bis(phosphorodithioate)), endothion (S-5-methoxy-4-oxo-4H-pyran-2-ylmethyl O,O-dimethyl phosphorothioate), menazon (S-4,6-diamino-1,3,5-triazin-2-ylmethyl O,O-dimethyl phosphorodithioate), morphothion (O,O-dimethyl S-morpholinocarbonylmethyl phosphorodithioate), phosalone (S-6-chloro-2,3-dihydro-2-oxobenzoxazol-3-ylmethyl O,O-diethyl phosphorodithioate), pyraclofos ((RS)-O-1-(4-chlorophenyl)pyrazol-4-yl O-ethyl S-propyl phosphorothioate), pyridaphenthion (O-(1,6-dihydro-6-oxo-1-phenylpyridazin-3-yl) O,O-diethyl phosphorothioate), and quinothion (O,O-diethyl O-2-methylquinol-4-yl phosphorothioate); benzothiopyran organothiophosphate pesticides include: dithicrofos (S-(6-chloro-3,4-dihydro-2H-1-benzothi-in-4-yl) O,O-diethyl phosphorodithioate) and thicrofos (S-(6-chloro-3,4-dihydro-2H-1-benzothi-in-4-yl) O,O-diethyl phosphorothioate); benzotriazine organothiophosphate pesticides such as: azinphos-ethyl (S-(3,4-dihydro-4-oxobenzo[d]-[1,2,3]-triazin-3-ylmethyl) O,O-diethyl phosphorodithioate) and azinphos-methyl (S-(3,4-dihydro-4-oxobenzo[d]-[1,2,3]-triazin-3-ylmethyl) O,O-dimethyl phosphorodithioate); isoindole organothiophosphate pesticides include: dialifos, ((RS)—S-2-chloro-1-phthalimidoethyl O,O-diethyl phosphorodithioate) and phosmet (O,O-dimethyl S-phthalimidomethyl phosphorodithioate); isoxazole organothiophosphate pesticides such as: isoxathion (O,O-diethyl O-5-phenyl-1,2-oxazol-3-yl phosphorothioate) and zolaprofos ((RS)-O-ethyl S-3-methyl-1,2-oxazol-5-ylmethyl S-propyl phosphorodithioate); pyrazolopyrimidine organothiophosphate pesticides such as: chlorprazophos (O-(3-chloro-7-methylpyrazolo[1,5-a]pyrimidin-2-yl) O,O-diethyl phosphorothioate) and pyrazophos (ethyl 2-diethoxyphosphinothioyloxy-5-methylpyrazolo[1,5-a]pyrimidine-6-carboxylate); pyridine organothiophosphate pesticides including: chlorpyrifos (O,O-diethyl O-3,5,6-trichloro-2-pyridyl phosphorothioate) and chlorpyrifos-methyl (O,O-dimethyl O-3,5,6-trichloro-2-pyridyl phosphorothioate); pyrimidine organothiophosphate pesticides such as: butathiofos (O-2-tert-butylpyrimidin-5-yl O,O-diethyl phosphorothioate), diazinon (O,O-diethyl O-2-isopropyl-6-methylpyrimidin-4-yl phosphorothioate), etrimfos (O-6-ethoxy-2-ethylpyrimidin-4-yl O,O-dimethyl phosphorothioate), lirimfos (O-6-ethoxy-2-isopropylpyrimidin-4-yl O,O-dimethyl phosphorothioate), pirimiphos-ethyl (O-2-diethylamino-6-methylpyrimidin-4-yl O,O-diethyl phosphorothioate), pirimiphos-methyl (O-2-diethylamino-6-methylpyrimidin-4-yl O,O-dimethyl phosphorothioate), primidophos (O,O-diethyl O-2-N-ethylacetamido-6-methylpyrimidin-4-yl phosphorothioate), pyrimitate (O-2-dimethylamino-6-methylpyrimidin-4-yl O,O-diethyl phosphorothioate), and tebupirimfos ((RS)—O-(2-tert-butylpyrimidin-5-yl) O-ethyl O-isopropyl phosphorothioate); quinoxaline organothiophosphate pesticides illustratively including: quinalphos (O,O-diethyl O-quinoxalin-2-yl phosphorothioate) and quinalphos-methyl (O,O-dimethyl O-quinoxalin-2-yl phosphorothioate); thiadiazole organothiophosphate pesticides such as: athidathion (O,O-diethyl S-2,3-dihydro-5-methoxy-2-oxo-1,3,4-thiadiazol-3-ylmethyl phosphorodithioate), lythidathion (S-5-ethoxy-2,3-dihydro-2-oxo-1,3,4-thiadiazol-3-yhnethyl O,O-dimethyl phosphorodithioate), methidathion (S-2,3-dihydro-5-methoxy-2-oxo-1,3,4-thiadiazol-3-ylmethyl O,O-dimethyl phosphorodithioate) or (3-dimethoxyphosphinothioylthiomethyl-5-methoxy-1,3,4-thiadiazol-2(3H)-one), and prothidathion (S-2,3-dihydro-5-isopropoxy-2-oxo-1,3,4-thiadiazol-3-ylmethyl O,O-diethyl phosphorodithioate); triazole organothiophosphate pesticides such as: isazofos (O-5-chloro-1-isopropyl-1H-1,2,4-triazol-3-yl O,O-diethyl phosphorothioate) and triazophos (O,O-diethyl O-1-phenyl-1H-1,2,4-triazol-3-yl phosphorothioate); phenyl organothiophosphate pesticides including: azothoate (O-4-(4-chlorophenylazo)phenyl O,O-dimethyl phosphorothioate), bromophos (O-4-bromo-2,5-dichlorophenyl O,O-dimethyl phosphorothioate), bromophos-ethyl (O-4-bromo-2,5-dichlorophenyl O,O-diethyl phosphorothioate), carbophenothion (S-4-chlorophenylthiomethyl O,O-diethyl phosphorodithioate), chlorthiophos (isomeric reaction mixture in which O-2,5-dichlorophenyl-4-methylthiophenyl O,O-diethyl phosphorothioate predominates), cyanophos (O-4-cyanophenyl O,O-dimethyl phosphorothioate), cythioate (O,O-dimethyl O-4-sulfamoylphenyl phosphorothioate), dicapthon (O-2-chloro-4-nitrophenyl O,O-dimethyl phosphorothioate), dichlofenthion, O-2,4-dichlorophenyl O,O-diethyl phosphorothioate), etaphos ((RS)—O-2,4-dichlorophenyl O-ethyl S-propyl phosphorothioate), famphur (O-4-dimethylsulfamoylphenyl O,O-dimethyl phosphorothioate), fenchlorphos (O,O-dimethyl O-2,4,5-trichlorophenyl phosphorothioate), fenitrothion (O,O-dimethyl O-4-nitro-m-tolyl phosphorothioate), fensulfothion (O,O-diethyl O-4-methylsulfinylphenyl phosphorothioate), fenthion (O,O-dimethyl O-4-methylthio-m-tolyl phosphorothioate), fenthion-ethyl (O,O-diethyl O-4-methylthio-m-tolyl phosphorothioate), heterophos ((RS)—O-ethyl O-phenyl S-propyl phosphorothioate), jodfenphos (O-2,5-dichloro-4-iodophenyl O,O-dimethyl phosphorothioate), mesulfenfos (O,O-dimethyl O-4-methylsulfinyl-m-tolyl phosphorothioate), parathion (O,O-diethyl O-4-nitrophenyl phosphorothioate), parathion-methyl (O,O-dimethyl O-4-nitrophenyl phosphorothioate), phenkapton (S-2,5-dichlorophenylthiomethyl O,O-diethyl phosphorodithioate), phosnichlor (O-4-chloro-3-nitrophenyl O,O-dimethyl phosphorothioate), profenofos ((RS)—O-4-bromo-2-chlorophenyl O-ethyl S-propyl phosphorothioate), prothiofos ((RS)—O-2,4-dichlorophenyl O-ethyl S-propyl phosphorodithioate), sulprofos ((RS)—O-ethyl O-4-(methylthio)phenyl S-propyl phosphorodithioate), temephos (O,O,O′,O′-tetramethyl O,O′-thiodi-p-phenylene bis(phosphorothioate)), trichlormetaphos-3 ((RS)—O-ethyl O-methyl O-2,4,5-trichlorophenyl phosphorothioate), and trifenofos ((RS)—O-ethyl S-propyl O-2,4,6-trichlorophenyl phosphorothioate); phosphonate pesticides such as: butonate ((RS)-2,2,2-trichloro-1-(dimethoxyphosphinoyl)ethyl butyrate) and trichlorfon (dimethyl (RS)-2,2,2-trichloro-1-hydroxyethylphosphonate); phosphonothioate pesticides such as: mecarphon (methyl (RS) [methoxy(methyl) phosphinothioylthio]acetyl (methyl) carbamate); phenyl ethylphosphonothioate pesticides including: fonofos ((RS)—O-ethyl S-phenyl ethylphosphonodithioate) and trichloronat ((RS)—O-ethyl O-2,4,5-trichlorophenyl ethylphosphonothioate); phenyl phenylphosphonothioate pesticides such as: cyanofenphos ((RS)—O-4-cyanophenyl O-ethyl phenylphosphonothioate), EPN ((RS)—O-ethyl O-4-nitrophenyl phenylphosphonothioate) and leptophos ((RS)—O-4-bromo-2,5-dichlorophenyl O-methyl phenylphosphonothioate); phosphoramidate pesticides including: crufomate ((RS)-4-tert-butyl-2-chlorophenyl methyl methylphosphoramidate), fenamiphos ((RS)-ethyl 4-methylthio-m-tolyl isopropylphosphoramidate), fosthietan (diethyl 1,3-dithietan-2-ylidenephosphoramidate), mephosfolan (diethyl 4-methyl-1,3-dithiolan-2-ylidenephosphoramidate), phosfolan (diethyl 1,3-dithiolan-2-ylidenephosphoramidate), and pirimetaphos ((RS)-2-diethylamino-6-methylpyrimidin-4-yl methyl methylphosphoramidate); phosphoramidothioate pesticides such as: acephate ((RS)—O,S-dimethyl acetylphosphoramidothioate), isocarbophos ((RS)—O-2-isopropoxycarbonylphenyl O-methyl phosphoramidothioate), isofenphos ((RS)—O-ethyl O-2-isopropoxycarbonylphenyl isopropylphosphoramidothioate), methamidophos ((RS)—O,S-dimethyl phosphoramidothioate), and propetamphos ((RS)-(E)-O-2-isopropoxycarbonyl-1-methylvinyl O-methyl ethylphosphoramidothioate); phosphorodiamide pesticides including: dimefox (tetramethylphosphorodiamidic fluoride), mipafox, N,N′-di-isopropylphosphorodiamidic fluoride), and schradan (octamethylpyrophosphoric tetra-amide).

Chemical warfare organophosphates are commonly called “organophosphate nerve agents” and include those exemplified in Ellison, D. H., Handbook of Chemical and Biological Warfare Agents, 1999, CRC Press. Specific examples of organophosphate nerve agents include phosphoramidocyanidic acid, dimethyl-, ethyl ester also known as GA and Tabun; phosphonofluoridic acid, methyl-, 1-methylethyl ester also known as GB and Sarin; phosphonofluoridic acid, methyl-, 1,2,2-trimethylpropyl ester also known as GD and Soman; phosphonofluoridic acid, ethyl-, 1-methylethyl ester also known as GE; phosphonofluoridic acid, methyl-, cyclohexyl ester also known as GF and cyclosarin; phosphoramidofluoridic acid, dimethyl-, 2-(dimethylamino)ethyl ester also known as GV and GP; phosphonothioic acid, ethyl-, S-[2-(diethylamino)ethyl]O-ethyl ester also known as VE; phosphorothioic acid, S-[2-(diethylamino)ethyl]O,O-diethyl ester also known as VG and Amiton; phosphonothioic acid, methyl-, S-[2-(diethylamino)ethyl]O-ethyl ester also known as VM; phosphonothioic acid, methyl-, S-[2-(diethylamino)ethyl]O-(2-methylpropyl) ester also known as VRm RVX, and Russian VX; phosphonothioic acid, ethyl-, S-[2-[bis(1-methylethyl)amino]ethyl]O-ethyl ester also known as VS; and phosphonothioic acid, methyl-, S-[2-[bis(1-methylethyl)amino]ethyl]O-ethyl ester also known as VX.

A therapeutically effective amount is that amount which decreases a symptom or sign of organophosphate-induced cardiac abnormalities. Exemplary symptoms and signs include arrhythmia, fibrillation, abnormal ECG readout such as changes in the P-wave, the depolarization of the atria, and prominent modulation of the T-wave, rising resting potential, Brugada-like symptoms, an initial bradycardia, Torsade de Pointes and tachycardia. Such signs and symptoms are monitored by methods typically used to monitor cardiac parameters, such as ECG. Further symptoms and signs are described herein. In some cases, symptoms and signs associated with intoxication are not specifically associated with toxin exposure. In such cases, intoxication may be confirmed by tests for presence of the toxin, a metabolite of the toxin, a degradation product of the toxin and/or a personal history indicative of likelihood of exposure to the toxin.

An inventive method includes treatment of disorders associated with organophosphate toxicity, particularly cardiac abnormalities such as heart failure caused by ventricular fibrillation due to organophosphate-toxicity and cardiogenic shock due to organophosphate-toxicity.

One characteristic of organophosphate intoxication is distortion of ion gradients across excitable membrane cell membranes, particularly cardiac cell membranes. Thus, an inventive method includes administering a compound to modulate organophosphate-induced activation and/or inhibition of one or more cardiac cell transmembrane ion currents, so as to inhibit organophosphate-induced distortion of the action potential of myocytes, support restoration of usual intracellular ionic concentrations, and support an increase in cardiac contractility.

The present invention provides methods for modulation of selected membrane currents to aid in inhibiting organophosphate-induced distortion of the action potential of myocytes, support restoration of usual intracellular ionic concentrations, and support an increase in cardiac contractility. It has been found by the present inventors that modeling inhibition of an organophosphate-induced activation of a membrane current which is typically swelling-activated has such beneficial effects on modeled cell parameters.

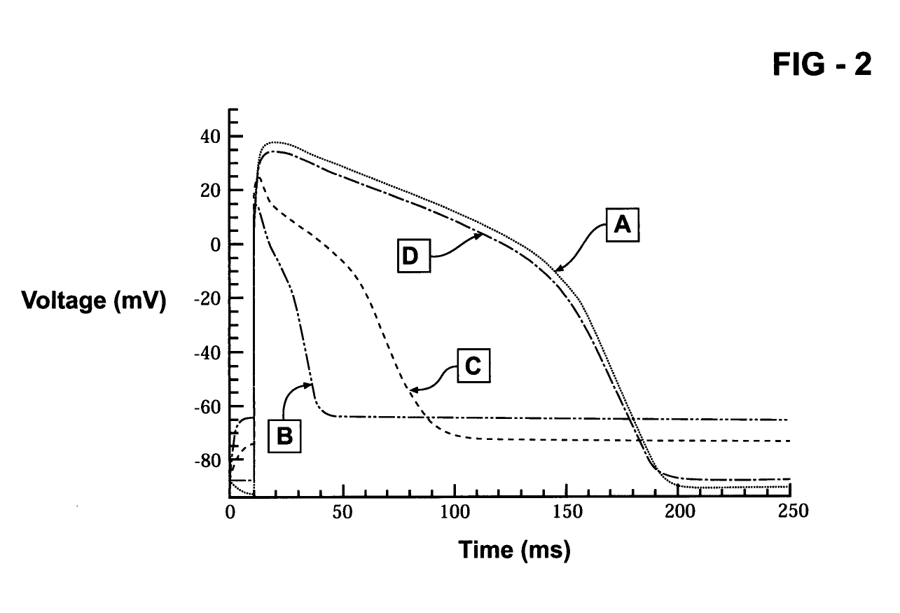

In a particular example, modeled inhibition of a transmembrane chloride current activated in an organophosphate exposed cell or tissue aids in reestablishing characteristics of normal cell and tissue functionality, for instance by inhibiting organophosphate-induced distortion of the action potential of myocytes, supporting restoration of usual intracellular ionic concentrations, and supporting an increase in cardiac contractility. FIG. 2 illustrates a model of organophosphate intoxication of cardiac cells and the effect of terminating the cell membrane chloride current. A normal action potential typical of cardiac tissue unaffected by organophosphate is shown in the rightmost trace (A) representing a control. The leftmost trace (B) shows a model of an action potential of an organophosphate affected cardiac ventricular cell. Note the shortened cycle length, the raised equilibrium potential and the reduced amplitude. Restoring the usual potassium ion concentrations across the cell membrane helps, as shown in (C), but the cycle length, a measure of the tachycardia, is still less than half of the control condition. Antagonizing the organophosphate-caused chloride membrane current, as shown in the trace (D) restores the normal action potential, indicating restored membrane potential and contractility.

Thus, compounds administered in a method according to the invention include those that inhibit usually inactive membrane currents activated following exposure of tissue to organophosphate.

In one embodiment, an inventive method of treating organophosphate poisoning in an individual subject includes administering a pharmaceutical composition which contains a therapeutically effective amount of a chloride channel modulator to an individual subject in need thereof.

In a particular embodiment, the chloride channel modulator is an antagonist of a chloride current activated in a cell exposed to organophosphate. In a preferred embodiment, the chloride current modulator is a modulator of a chloride current known as “I_{Cl,swell}.” I_{Cl,swell}, also known as “I_{Cl,vol},” is a cell volume regulated chloride current present in cardiac cells. See, for example, Hume, J. R. et al., Physiol. Reviews, 80:31-81, 2000 and Lang, F. et al., Physiol. Reviews, 78:247-306, 1998.

Chloride current antagonists illustratively include disulfonic stilbenes, arylaminobenzoates, fenamates, anthracene carboxylates, indanylalkanoic acids, clofibric acid, clofibric acid derivatives, sulfonylureas, calixarenes, suramin, and tamoxifen.

Particular chloride conductance modulators administered in an embodiment of an inventive method include inhibitors of an “I_{Cl,swell}” chloride current. For example, a preferred disulfonic stilbene inhibitor of an “I_{Cl,swell}” chloride current included in a method and composition according to the invention is 4,4′-diisothiocyanostilbene-2,2′-disulfonic acid (DIDS). In another embodiment, disulfonic stilbenes included in a method and composition according to the invention illustratively include 4,4′-dinitrostilbene-2,2′-disulfonic acid (DNDS) and 4-acetamindo-4′-isothiocyanostilbene-2,2′-disulfonic acid (SITS).

In another embodiment, an “I_{Cl,swell}” chloride conductance inhibitor included in an inventive method and composition is tamoxifen. In further embodiments, an “I_{Cl,swell}” chloride conductance inhibitor included in an inventive method and composition is illustratively 5-nitro-2-(3-phenylpropylamino) benzoic acid (NPPB); niflumic acid (NFA); flufenamic acid; anthracene-9-carboxylate (9AC); diphenylaminecarboxylate (DPC); 2-(p-chlorophenoxy)propionic acid (CPP—a clofibric acid derivative); and indanyloxyacetic acid (IAA-94).

An inhibitor of “I_{Cl,swell}” chloride current may be identified using methods known in the art. For example, one or more cells placed in a recording chamber may be exposed to a stimulus causing a change in cell volume. A chloride current activated in response to the change in cell volume may be detected by methods such as whole cell or patch clamp techniques. An inhibitor of “I_{Cl,swell}” chloride current may be identified as an agent that causes a specific decrease in “I_{Cl,swell}” chloride current.

A chloride conductance inhibitor included in a composition and method according to the invention preferably produces at least about a 50% decrease in an organophosphate exposure-induced membrane chloride current. Further, a chloride conductance inhibitor included in a composition and method according to the invention preferably induces at least about a 50% increase in cardiac action potential duration in an organophosphate-intoxicated substrate, that is, the tissue being acted on.

One consequence of organophosphate intoxication is deleterious accumulation of intracellular calcium. A chloride conductance inhibitor included in a composition and method according to the invention causes at least about a 1-12% decrease in intracellular calcium concentration and preferably causes at least about a 2-6% decrease in. intracellular calcium concentration. Such a decrease in intracellular calcium concentration may be measured in a standard in vitro calcium sensitizing assay, such as that detailed in M. Endoh, Mechanism of action of novel cardiotonic agents, J. Cardiovascular Pharmacology, 2002, 40:323-338.

Organophosphate poisoning is a toxicity caused by multiple effects of the organophosphate compounds on metabolic processes. Thus, cardiac cells are affected by organophosphate via several pathways and organophosphate-related cardiac pathology appears to be the result of such multiple effects. Some of the toxic effects of organophosphate occur by mechanisms such as the affinity of organophosphate compounds for acetylcholinesterase. Effects of acetylcholinesterase inhibition include stimulation of the aforementioned “I_{Cl,swell}” chloride conductance. In addition, organophosphate toxins block the fast outward rectifying potassium current with the subsidiary effect upon energy production at the cellular level.

A conductance modulator is optionally delivered in conjunction with a therapeutic agent in addition to a conductance modulator in one embodiment. A therapeutic agent may be included in a pharmaceutical composition with a conductance modulator and/or administered separately.

A composition including a chloride current modulator, particularly an inhibitor of I_{Cl,swell} and a therapeutic agent, is administered to inhibit organophosphate-induced distortion of the action potential of myocytes, support restoration of usual intracellular ionic concentrations, and support an increase in cardiac contractility. Exemplary therapeutic agents illustratively include a second current modulator, an antiarrhythmic drug, an acetylcholine receptor antagonist, an anticonvulsant, an organophosphate clearing agent, an inhibitor of protein kinase C, a modulator of a mitochondrial membrane moiety, and a combination thereof. Combinations of a chloride current modulator, particularly an inhibitor of I_{Cl, swell} and a therapeutic agent may have a synergistic effect in inhibiting organophosphate-induced distortion of the action potential of myocytes, supporting restoration of usual intracellular ionic concentrations, and supporting an increase in cardiac contractility. In particular, lesser amounts of a chloride current modulator may be necessary to achieve reduction in symptoms and signs of cardiac abnormality when the modulator is included in a composition with a therapeutic agent. Such a composition may therefore provide benefits of treatment of toxin-induced cardiac abnormality as well as cost savings and reduction in side effects. Further, lesser amounts of a therapeutic agent may be administered when included in an inventive composition.

An exemplary therapeutic agent suitable in this regard is an organophosphate clearing agent. The term “organophosphate clearing agent” as used herein is intended to mean a compound having a beneficial effect on an organophosphate-poisoned individual, particularly an effect of inhibiting the action of organophosphate in the affected individual such as by stimulating removal, sequestration, or metabolism of the organophosphate and/or regenerating acetylcholinesterase activity such that the toxic effects are inhibited.

An organophosphate clearing agent includes an oxime agent such as obidoxime, pralidoxime and asoxime, and salts or other derivatives thereof exemplified by asoxime chloride, pralidoxime chloride, pralidoxime methanesulfonate, known as the mesilate, and pralidoxime methyl sulphate.

Exemplary dosing and administration of organophosphate clearing agents includes intravenous administration of pralidoxime mesilate in amounts of about 20-50 mg/kg body weight every 4-6 hours, usually for about 3 days or less. Pralidoxime chloride may be administered intravenously in amounts of about 1 g every 4-6 hours. Effective levels in blood serum may be in the range of about 4-15 micrograms/milliliter. Pralidoxime iodide may be administered intravenously in amounts of about 20-50 milligrams/kg body weight intermittently to alleviate symptoms. Obidoxime may be given in amounts ranging from about 2-4 mg/kg body weight intravenously. Asoxime chloride may be given as about 250-1000 milligrams diluted in 1-5 milliliters of distilled water intramuscularly 4 times/day for 2-7 days. One of skill in the art will know how to adjust such doses in order to meet the demands of a particular therapeutic situation.

In a further example of a therapeutic agent, a second conductance modulator is optionally administered in order to inhibit organophosphate-induced distortion of the action potential of myocytes, support restoration of usual intracellular ionic concentrations, and support an increase in cardiac contractility. For example, more than one modulator of a chloride current may be administered. The multiple chloride current modulators optionally modulate the same or different chloride channels. In another example, such a compound is a sodium channel opener such as aconitine, and/or an agent such as veratridine which slows the sodium current inactivation, (Wang, G K and Wang S Y, Veratridine block of rat skeletal muscle Nav1.4 sodium channels in the inner vestibule, J Physiol., 2003, 548:667-675.), and/or a calcium channel opener such as BAY K 8644 and/or flecanide.

In a further embodiment, a second conductance modulator is administered to address the effects of organophosphate toxins in blocking the fast outward rectifying potassium current. For example, an inhibitor of an inward rectifier may be administered. An inhibitor of an inward rectifier inhibits K_{Ach}, a muscarinic potassium channel found in atrial myocytes, for example. Such an inhibitor is illustratively tertiapin, a peptide originally isolated from Apis mellifera honeybee venom. Tertiapin-Q is a synthetic version of the peptide in which a methionine is replaced by a glutamine to yield the peptide having the sequence: ALCNCNRIIIPHQCWKKCGKK. See Drici, M. D. et al., Br J Pharmacol. 2000, 131(3):569-77, for discussion of tertiapin effects on potassium channels in mammalian heart.

Another example of a therapeutic agent included in a method of treatment and/or a pharmaceutical composition according to the invention is an acetylcholine receptor antagonist. An acetylcholine receptor antagonist may be an antagonist of a nicotinic and/or muscarinic receptor. Muscarinic antagonists are preferred in one embodiment. Exemplary muscarinic acetylcholine receptor antagonists include scopolamine, ipratropium, methantheline, propantheline, tolterodine (tartrate), anisotropine, clidinium, dicyclomine, glycopyrrolate, homatropine, hyoscyamine, mepenzolate, methscopolamine, and pirenzepine. Atropine is a preferred muscarinic acetylcholine receptor antagonist.

An anticonvulsant may be administered in an inventive method and/or as part of a pharmaceutical composition of the invention. Preferred anticonvulsants include benzodiazepines such as diazepam, lorazepam, and midazolam, each given in amounts in the range of about 0.05-5 mg/kg body weight, preferably in the range of about 0.1-1 mg/kg body weight.

Another example of a therapeutic agent is an anti-arrhythmic drug administered in order to further support inhibition of organophosphate-induced distortion of the action potential of myocytes, restoration of usual intracellular ionic concentrations, and an increase in cardiac contractility. Antiarrhythmic drugs illustratively include class I antiarrhythmics which are sodium channel blockers such as class IA antiarrhythmics illustratively including quinidine (Quinidex), procainamide (Pronestyl) and disopyramide (Norpace); class IB antiarrhythmics illustratively including lidocaine (Xylocaine), tocainide (Tonocard), and mexiletine (Mexitil); class IC antiarrhythmics illustratively including encainide (Enkaid), and flecainide (Tambocor); class II antiarrhythmics which are beta blockers illustratively including propranolol (Inderal), acebutolol (Sectral), esmolol (Brevibloc), metoprolol, and atenolol; class III antiarrhythmics which are potassium channel blockers illustratively including sotalol (Betapace), dofetilide and amiodarone (Cordarone) and class IV antiarrhythmics which are calcium channel blockers illustratively including verapamil (Calan, Isoptin), diltiazem (Cardizem) and mebefradil (Posicor). In addition, some antiarrhythmics such as alinidine may act as chloride channel blockers, see Millar J S and Williams E M., Pacemaker selectivity: influence on rabbit atria of ionic environment and of alinidine, a possible anion antagonist. Cardiovasc. Res. 1981, 15(6):335-50. Digoxin is a further antiarrhythmic optionally administered. Dosage of such agents is known in the art and is illustrated in standard texts such as R. N. Fogoros, Antiarrhythmic Drugs: A Practical Guide, Blackwell Publishers, 1997, and Mosby's Drug Consult, 2005, Mosby Inc., Elsevier, St. Louis Mo., ISBN 0-323-03393-8. For example, quinidine gluconate is typically administered in doses ranging from about 5-10 mg/kg (Mosby's Drug Consult p.II-2466-2475); procainamide is generally given in amounts of up to 50 mg/kg/day (Mosby's Drug Consult p.II-2409-2412); disopyramide is administered in amounts ranging from about 400-1200 mg/day (Mosby's Drug Consult p.II-852-855); lidocaine is generally given in amounts in the range of 50-100 mg administered intravenously (Mosby's Drug Consult p.II-1744-1751); tocainide is usually given in amounts ranging from 1200-1800 mg/day (Mosby's Drug Consult p.II-2833); mexiletine is generally given in amounts of about 600-1200 mg/day (Mosby's Drug Consult p.II-1951-1953); flecainide is usually administered in amount ranging from about 100-300 mg/day (Mosby's Drug Consult p.II-1189-1190); propranolol is typically given in amounts in the range of about 80-640 mg/day (Mosby's Drug Consult p.II-2445-2451); acebutolol is generally given in amounts in the range of about 200-1200 mg/day (Mosby's Drug Consult p.II-13-14); esmolol is typically administered in amounts ranging from about 50-500 micrograms/kg/min (Mosby's Drug Consult p.II-1019-1022); sotalol is generally given in amounts ranging from 160-640 mg/day (Mosby's Drug Consult p.II-2642-2650); dofetilide is generally given in amounts ranging from 250-1000 micrograms/day (Mosby's Drug Consult p.II-872-876); amidarone is usually administered in an amount in the range of about 400-1600 mg/day in oral form (Mosby's Drug Consult p.II-121-130); verapamil is typically given in an amount in the range of about 120-480 mg/day (Mosby's Drug Consult p.II-2977-2983); digoxin is administered as described in Mosby's Drug Consult p.II-810-819; and diltiazem is generally administered in amounts in the range of about 180-360 mg/day in oral formulation (Mosby's Drug Consult p.II-827-839).

In one embodiment, a therapeutic agent is administered which addresses effects of organophosphate intoxication on mitochondrial function. As noted above, one of the effects of organophosphate intoxication is inhibition of oxygen metabolism. Consequences of such inhibition include distortion of usual ion concentrations across the mitochondrial membrane. For example, a particular problem is accumulation of calcium in mitochondria, due in part to insufficient production of ATP necessary to sustain activity of mechanisms for maintenance of normal calcium concentrations, such as the Na⁺-Ca⁺⁺ exchanger. Further, sodium and potassium concentrations in mitochondria are distorted due, at least in part, to the decreased production of ATP and consequent inhibition of a Na⁺-K⁺ pump. A further deleterious effect of organophosphate intoxication on cells and mitochondria is increased generation of reactive oxygen species.

A therapeutic agent which addresses effects of organophosphate intoxication on mitochondrial function includes an agonist or antagonist of a mitochondrial membrane channel ion exchanger and/or pump effective to normalize distorted ion concentrations across the mitochondrial membrane. The mitochondrial membrane moiety may be selected from the group consisting of: an ion channel, an ion pump, an ion exchanger. Such compounds may be included to improve mitochondrial energy production, dampen mitochondrial calcium accumulation and reactive oxygen species production. For example, since calcium concentrations inside the mitochondria are typically increased following organophosphate exposure, an agent may be administered which is effective to activate or enhance an outward movement of calcium from a mitochondrion. Exemplary agents effective to stimulate calcium efflux include menadione (Henry T R et al., J. Toxicol. Environ. Health, 45(4):489-504, 1995).

In other examples, an agonist or antagonist of a mitochondrial membrane channel and/or pump stimulates activity of a mitochondrial membrane potassium channel, such as mitoKATP and/or mitoKCa. A particular compound which stimulates mitoKATP is diazoxide.

Optionally further included is administration of a therapeutic agent which is a protein kinase C (PKC) inhibitor. In the presence of organophosphate, rising inorganic phosphate (Pi) and declining phosphocreatine (PCr) are present in cells, affecting the electrophysiology of the tissue. An exemplary PKC inhibitor which may be used is H-7 (1-(5-isoquinolinesulfonyl)-2-methylpiperazine, an isoquinolinesulfonamide), a pharmacological inhibitor of PKC which prevents and/or inhibits the changes in the PCr and Pi.

Preferably, a patient is identified and selected for treatment according to an inventive method. For example, a patient in need of treatment may be an individual that is suffering from organophosphate intoxication such that an increase in myocardial contractility with reduced energy requirements is an intended therapy and where inhibition of organophosphate-induced distortion of the action potential of myocytes and support of restoration of usual intracellular ionic concentrations is desirable.

As noted above, organophosphate toxins are in common use in agricultural settings, as well as being a hazard encountered on the battlefield or in terrorist situations. Thus, an individual in need of treatment of organophosphate poisoning may be characterized by an occupation likely to bring the individual in contact with organophosphate and/or history of organophosphate exposure. Often of interest in identifying an individual in need of treatment is a history of the individual's recent activity or location, presence in a war zone or area known for terrorist activity being an indicator making exposure to organophosphate more likely.

Organophosphate poisoning may occur by any of various routes illustratively including ingestion, inhalation, skin exposure, mucosal exposure, and parenteral exposure. Organophosphate exposure leading to toxic effects may be in any of various forms such as exposure to organophosphate containing gases or liquids.

Symptoms and signs of organophosphate poisoning include early symptoms such as anxiety, headache, vertigo, hyperpnea, dyspnea, hypertension, bradycardia and cardiac arrhythmias such as sinus or AV nodal arrhythmias. Further symptoms include loss of consciousness, convulsions and cardiac arrest. Of particular interest in the context of an embodiment of an inventive method are cardiovascular symptoms including electrocardiography changes such as atria fibrillation, entopic ventricular heartbeats, abnormal QRS complex and sinus bradycardia. Elevated blood organophosphate concentration and decreased acetylcholinesterase activity can confirm exposure to organophosphate. However, where likelihood of organophosphate exposure is present, rapid treatment is required since death can occur in less than 10 minutes in severe cases. Thus, in one embodiment, an individual selected for treatment with an inventive composition and/or method is suffering from organophosphate-induced heart-failure and/or an organophosphate-induced contractility deficit. Such an individual may have a history or likelihood of organophosphate exposure and symptoms of organophosphate-induced heart failure and/or an organophosphate-induced contractility deficit. Optionally, organophosphate exposure is confirmed by assay for organophosphate, an organophosphate metabolite, a clinical and/or pathophysiological finding consistent with organophosphate exposure, especially by acetylcholinesterase activity assay.

The methods of the invention include both acute and chronic therapies. Relatively long-term administration of a therapeutic agent also will be beneficial after a patient has suffered from chronic organophosphate-caused heart failure to provide increased exercise tolerance and functional capacity. For example, a chloride conductance inhibitior, such as DIDS, can be administered to a patient after having suffered heart failure due to organophosphate caused toxicity to promote enhanced functional capacity.

For example, DIDS can be immediately administered to a patient, e.g. intravenously or intraperitonially, that has suffered or is suffering from congestive heart failure or cardiogenic shock. Such immediate administration preferably would entail administration of a therapeutic agent within minutes after a subject exhibits a symptom and/or sign of organophosphate-caused ventricular fibrillation or cardiogenic shock. In one embodiment, an oral dosage is preferred.

A patient in need of treatment for organophosphate poisoning is discussed herein in general terms relating to human individuals. However, inventive methods and compositions for individuals of other species, particularly mammalian species such as non-human primates, sheep, dogs, cats, cattle, pigs, horses and the like, are considered to be within the scope of the invention.

While compositions and methods according to the invention are described as relating to treatment of organophosphate-induced cardiac symptoms and signs, it is appreciated that other cells and tissues are affected by organophosphate and that compositions and methods described herein may be applicable to treatment of those other cells and tissues. In particular, excitable membranes of cells such as neurons may have membrane potentials and ion concentrations across those membranes are distorted as a result of organophosphate intoxication and it is appreciated that inventive methods and compositions may be used to treat such sequelae of organophosphate exposure.

An inventive pharmaceutical composition includes a modulator of a biological cell membrane conductance as described herein and a pharmaceutically acceptable carrier. The term “pharmaceutically acceptable carrier” as used herein is intended to refer to a carrier or diluent that is generally non-toxic to an intended recipient and which does not significantly inhibit activity of an active agent included in the composition.

An inventive composition is suitable for administration to patients by a variety of systemic and/or local routes illustratively including intravenous, oral, parenteral, intrathecal, intraventricular, intracardiac, pericardiac, and mucosal.

An inventive composition may be administered acutely or chronically. For example, in an emergency situation, a conductance modulator included in a composition as described herein may be administered as a unitary dose or in multiple doses over a relatively limited period of time, such as seconds-hours. In a further embodiment, administration may include multiple doses administered over a period of days-years, such as for chronic treatment of long-lasting sequelae of organophosphate poisoning.

A therapeutically effective amount of a current modulator and of a therapeutic agent described herein will vary independently depending on the particular compound and on the particular agent used, the severity of the toxin exposure including the route of toxin exposure and the identity of the toxin, the length of time since toxin exposure and the general physical characteristics of the individual to be treated. One of skill in the art could determine a therapeutically effective amount in view of these and other considerations typical in medical practice. In general it is contemplated that a therapeutically effective amount would be in the range of about 0.001 mg/kg-100 mg/kg body weight, more preferably in the range of about 0.01-10 mg/kg, and further preferably in the range of about 0.1-5 mg/kg. Further, dosage may be adjusted depending on whether treatment is to be acute or continuing.

Compositions suitable for delivery may be formulated in various forms illustratively including physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, and vehicles include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of the required particle size in the case of dispersions, and by the use of surfactants, such as sodium lauryl sulfate. Such formulations are administered by a suitable route including parenteral and oral administration. Administration may include systemic or local injection, and particularly intravenous injection.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and substances similar in nature. Prolonged delivery of an injectable pharmaceutical form can be brought about by the use of agents delaying absorption, for example, aluminum monostearate and gelatin.

Solid dosage forms for oral administration include capsules, tablets, pills, powders, and granules. In such solid dosage forms, a conductance modulator is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, plant starches such as potato or tapioca starch, a1 ginic acid, certain complex silicates, and sodium carbonate, (e) solution retarders, as for example, paraffin, (f) absorption accelerators, as for example, quaternary ammonium compounds, (g) wetting agents, as for example, cetyl alcohol, glycerol monostearate, and glycols (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied with to a thickness such that the entire coating does not dissolve in the gastrointestinal fluids at pH below 3 associated with stomach acids, yet dissolves above pH 3 in the small intestine environment. It is expected that any anionic polymer exhibiting a pH-dependent solubility profile is readily used as an enteric coating in the practice of the present invention to achieve delivery of the active to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particularly preferred enteric coating material for use herein is those acrylic acid polymers and copolymers available under the trade name EUDRAGIT®, Roehm Pharma (Germany). The EUDRAGIT® series L, L-30D S copolymers, and cross-linked polymers, see for example U.S. Pat. No. 6,136,345, are most preferred since these are insoluble in stomach and dissolve in the intestine.

The enteric coating provides for controlled release of the active agent, such that release is accomplished at a predictable location in the lower intestinal tract below the point at which drug release would occur absent the enteric coating. The enteric coating also prevents exposure of the active agent and carrier to the epithelial and mucosal tissue of the buccal cavity, pharynx, esophagus, and stomach, and to the enzymes associated with these tissues. The enteric coating therefore helps to protect the active agent and a patient's internal tissue from any adverse event prior to drug release at the desired site of delivery. Furthermore, the coated solid dosages of the present invention allow optimization of drug absorption, active agent protection, and safety. Multiple enteric coatings targeted to release the active agent at various regions in the lower gastrointestinal tract would enable even more effective and sustained improved delivery throughout the lower gastrointestinal tract.

The enteric coating optionally contains a plasticizer to prevent the formation of pores and cracks that allow the penetration of the gastric fluids into the solid dosage. Suitable plasticizers illustratively include, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The enteric coating is applied to a solid dosage using conventional coating methods and equipment. For example, an enteric coating can be applied to a solid dosage using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in L. V. Allen, Jr. et al., Ansel's Pharmaceutical Dosage Forms and Drug Delivery Systems, 8th Ed. (Philadelphia, Pa.: Lippincott, Williams & Wilkins, 2004).

Liquid dosage forms for oral administration include a pharmaceutically acceptable carrier formulated as an emulsion, solution, suspension, syrup, or elixir. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to an inventive conjugate, may contain suspending agents, as for example, ethoxylated isostearyl alcohols, polyoxyethylene sorbitol and sorbitol esters, microcrystalline cellulose, aluminum metahydroxide, bentonite, agar-agar or tragacanth, or mixtures of these substances, and the like.

An ion channel modulator and/or therapeutic agent administered in an inventive method is defined herein as an ion channel modulator and/or therapeutic agent and pharmaceutically acceptable salts, derivatives, oxides and hydrates thereof. The term “pharmaceutically acceptable salt, derivative, oxide and hydrate” refers to a formulation that is substantially non-toxic to the individual being treated and which does not substantially inhibit the activity of an active agent being administered. The term “derivative” as used herein refers to a channel modulator and/or a therapeutic agent which is chemically modified to include a nitrogen, oxygen, carbon, sulfur, halogen or phosphorus containing moiety illustratively including a C₁-C₄ substituted or unsubstituted, straight chain or branched alkyl, an amine, a sulfhydryl and an oxide. The term “oxide” as used herein refers to an oxygen containing derivative of a channel modulator and/or a therapeutic agent. Exemplary oxygen containing derivatives include an oxygen containing moiety such as a carboxyl, a carbonyl, a sulfonyl, a sulfoxy, a hydroxyl, a nitro, a phosphate, and a C₁-C₄ substituted or unsubstituted, straight chain or branched alkyl linked to the channel modulator and/or a therapeutic agent by ester or ether linkage.

Further examples and details of pharmacological formulations and ingredients are found in standard references such as: A. R. Gennaro, Remington: The Science and Practice of Pharmacy, Lippincott Williams & Wilkins, 20th ed. (2003); L. V. Allen, Jr. et al., Ansel's Pharmaceutical Dosage Forms and Drug Delivery Systems, 8th Ed. (Philadelphia, Pa.: Lippincott, Williams & Wilkins, 2004); J. G. Hardman et al., Goodman & Gilman's The Pharmacological Basis of Therapeutics, McGraw-Hill Professional, 10th ed. (2001).

While compositions and methods described herein are primarily described in the context of organophosphate intoxication, it is appreciated that compositions and methods as described may be applicable to treatment of other types of toxic agent exposure in which a dormant ion current is activated following exposure such that cell membrane potentials and ion concentrations across those membranes are distorted, symptoms and signs indicative of pathological excitable cell function. Further, an individual may be exposed to combinations of toxic agents for which inventive methods and compositions are applicable.

Embodiments of inventive compositions and methods are illustrated in the following examples. These examples are provided for illustrative purposes and are not considered limitations on the scope of inventive compositions and methods.

### EXAMPLE 1

An individual having symptoms of organophosphate-induced cardiac arrhythmia is injected intravenously with 10 mg/kg 4,4′-diisothiocyanostilbene-2,2′-disulfonic acid (DIDS) dissolved in normal saline. The effect of the treatment is monitored by ECG.

### EXAMPLE 2

An individual presents with. symptoms of organophosphate-induced cardiac arrhythmia. A solution of 20 mg/kg of indanyloxyacetic acid (IAA-94) is prepared by dissolving the IAA-94 in ethanol and then diluting the material to the final concentration in normal saline. The organophosphate affected individual is injected intravenously with the IAA-94 preparation. The effect of the treatment may be monitored by ECG.

### EXAMPLE 3

An individual having symptoms of organophosphate-induced cardiac arrhythmia is injected intravenously with 0.5 mg/kg of tamoxifen dissolved in ethanol and then diluted to final concentration in normal saline. The effect of the treatment may be monitored by ECG.

### EXAMPLE 4

An individual having symptoms of organophosphate-induced cardiac arrhythmia is injected intravenously with 1 mg/kg of 5-nitro-2-(3-phenylpropylamino)benzoic acid (NPPB) dissolved in ethanol and then diluted to final concentration in normal saline. The effect of the treatment may be monitored by ECG.

### EXAMPLE 5

An individual having a history of organophosphate exposure and symptoms of cardiac rhythm abnormalities ingests 25 mg/kg of 4,4′-diisothiocyanostilbene-2,2′-disulfonic acid (DIDS) in tablet form. The effect of the treatment may be monitored by ECG.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of treating an organophosphate toxin-caused cardiac abnormality in an individual subject, comprising:
administering a pharmaceutical composition comprising a therapeutically effective amount of a chloride current modulator to an individual subject having a cardiac abnormality caused by intoxication with an organophosphate toxin, the chloride current modulator effective to modulate a chloride conductance, thereby reducing a symptom or sign of a toxin-caused cardiac abnormality and treating the toxin-induced cardiac abnormality.

2. The method of claim 1 wherein the chloride current modulator is a modulator of IC1, swell.

3. The method of claim 1 wherein the chloride current modulator is selected from the group consisting of: a disulfonic stilbene, an arylaminobenzoate, a fenamate, an anthracene carboxylate, an indanylalkanoic acid, clofibric acid, a clofibric acid derivative, a sulfonylurea, a calixarene, suramin, and tamoxifen.

4. The method of claim 2 wherein the modulator of IC1, swell is selected from the group consisting of: 4,4′-diisothiocyanostilbene-2,2′-disulfonic acid (DIDS); 4,4′-dinitrostilbene-2,2′-disulfonic acid (DNDS); 4-acetamindo-4′-isothiocyanostilbene-2,2′-disulfonic acid (SITS); tamoxifen; 5-nitro-2-(3-phenylpropylamino) benzoic acid (NPPB); niflumic acid (NFA); flufenamic acid; anthracene-9-carboxylate (9AC); diphenylaminecarboxylate (DPC); 2-(p-chlorophenoxy)propionic acid (CPP); and indanyloxyacetic acid (IAA-94).

5. The method of claim 1 further comprising administering a therapeutic agent to inhibit organophosphate-induced distortion of the action potential of myocytes, support restoration of usual intracellular ionic concentrations, and support an increase in cardiac contractility.

6. The method of claim 5 wherein the therapeutic agent is a second current modulator.

7. The method of claim 5 wherein the therapeutic agent is an antiarrhythmic drug.

8. The method of claim 5 wherein the therapeutic agent is a modulator of a mitochondrial membrane moiety selected from the group consisting of: an ion channel, an ion pump, an ion exchanger, and a combination thereof.

9. The method of claim 5 wherein the therapeutic agent is an inhibitor of protein kinase C.

10. The method of claim 5 wherein the therapeutic agent is an anticonvulsant.

11. The method of claim 5 wherein the therapeutic agent is an organophosphate clearing agent.

12. The method of claim 11 wherein the organophosphate clearing agent is selected from the group consisting of: obidoxime, asoxime, pralidoxime, a salt thereof; and a combination thereof.

13. The method of claim 1 wherein the cardiac abnormality is ventricular fibrillation.

14. The method of claim 1 wherein the cardiac abnormality is cardiogenic shock.

15. The method of claim 6 wherein the second current modulator is an inhibitor of a muscarinic potassium channel.

16. The method of claim 15 wherein the inhibitor of a muscarinic potassium channel is selected from the group consisting of: tertiapin, tertiapin-Q, and a combination thereof.

17. The method of claim 5 wherein the therapeutic agent is an acetylcholine receptor antagonist.

18. A pharmaceutically effective amount of a composition consisting of:
a chloride current modulator of IC1 swell; and
a combination therapeutic agent limited to the class of agents that in a single dose inhibit organophosphate-caused distortion of the action potential of myocytes, support restoration of usual intracellular ionic concentrations, and support an increase in cardiac contractility.

19. (canceled)

20. The composition of claim 18 wherein the therapeutic agent is selected from the group consisting of: a second current modulator, an antiarrhythmic drug, an acetylcholine receptor antagonist, an anticonvulsant, an organophosphate clearing agent, an inhibitor of protein kinase C, a modulator of a mitochondrial membrane moiety, and a combination thereof.
